(19) United States
(12) Patent Application Publication
(10) Pub. No.: US 2006/0074449 A1
(43) Pub. Date: Apr. 6, 2006

(54) Intravascular stimulation system with wireless power supply

(76) Inventors: Stephen Denker, Mequon, WI (US); Arthur J. Beutler, Greendale, WI (US)

Correspondence Address: QUARLES & BRADY LLP, 411 E. WISCONSIN AVENUE, SUITE 2040, MILWAUKEE, WI, 53202-4497, US

(21) Appl. No.: 11/199,030

(22) Filed: Aug. 8, 2005

Related U.S. Application Data
(63) Continuation-in-part of application No. 10/700,148, filed on Nov. 3, 2003, now Pat. No. 7,003,350.

(51) Int. Cl. A61N 1/05

(52) U.S. Cl. 607/2; 607/116

(57) ABSTRACT

An apparatus for stimulating tissue of a medical patient includes a power transmitter which periodically transmits a pulse of a radio frequency signal to a intravascular stimulator that is implanted in a vein or artery. The intravascular stimulator employs energy from the radio frequency signal to charge a storage device which serves as an electrical power supply. The intravascular stimulator also detects an electrical signal produced within the patient and responds thereto by applying a pulse of voltage from the storage device to a pair of electrodes implanted in the vascular system of the animal.

### CROSS-REFERENCE TO RELATED APPLICATIONS

This application is a continuation in part of U.S. patent application Ser. No. 10/700,148 filed on Nov. 3, 2003.

### STATEMENT REGARDING FEDERALLY SPONSORED RESEARCH OR DEVELOPMENT

Not Applicable

### BACKGROUND OF THE INVENTION

1. Field of the Invention

The present invention relates to implantable medical devices which deliver energy to stimulate tissue in an animal, and more particularly to transvascular stimulation in which the medical device is implanted in a vein or artery to stimulate the adjacent tissue or organ.

2. Description of the Related Art

A remedy for people with slowed or disrupted natural heart activity is to implant a cardiac pacing device which is a small electronic apparatus that stimulates the heart to beat at regular rates.

Typically the pacing device is implanted in the patient's chest and has sensor electrodes that detect electrical impulses associated with in the heart contractions. These sensed impulses are analyzed to determine when abnormal cardiac activity occurs, in which event a pulse generator is triggered to produce electrical pulses. Wires carry these pulses to electrodes placed adjacent specific cardiac muscles, which when electrically stimulated contract the heart chambers. It is important that the stimulation electrodes be properly located to produce contraction of the heart chambers.

Modern cardiac pacing devices vary the stimulation to adapt the heart rate to the patient's level of activity, thereby mimicking the heart's natural activity. The pulse generator modifies that rate by tracking the activity of the sinus node of the heart or by responding to other sensor signals that indicate body motion or respiration rate.

U.S. Pat. No. 6,445,953 describes a cardiac pacemaker that has a pacing device, which can be located outside the patient, to detect abnormal electrical cardiac activity. In that event, the pacing device emits a radio frequency signal, that is received by a circuit mounted on a stimulator body implanted in a vein or artery of the patient's heart. Specifically, the radio frequency signal induces a voltage pulse in an antenna and that pulse is applied across a pair of electrodes on the body, thereby stimulating adjacent muscles and contracting the heart. Although this cardiac pacing apparatus offered several advantages over other types of pacemakers, it required placement of sensing electrodes on the patient's chest in order for the external pacing device to detect when the heart requires stimulation.

### SUMMARY OF THE INVENTION

An apparatus is provided to electrically stimulate tissue or an organ of an animal. That apparatus includes a power transmitter which periodically transmits a pulse of a radio frequency signal to a intravascular stimulator that is implanted in a vein or artery of the animal.

The intravascular stimulator comprises a pickup device, such as a coil of wire for example, for receiving the radio frequency signal from the power transmitter and optionally an electrical signal produced within the animal, such as a signal emitted from the sinus node or muscle fibers of a heart. A stimulation signal circuit is connected to the pickup device and a pair of electrodes that are in contact with tissue of the animal and has an electrical storage device that is charged by electrical energy from the radio frequency signal. Upon being triggered, the stimulation signal circuit applies a voltage pulse across the pair of electrodes thereby stimulating the tissue of the animal adjacent the electrodes.

In a preferred embodiment of the intravascular stimulator, the stimulation signal circuit includes a discriminator and a pulse circuit. The discriminator is connected to the pickup device and controls charging of the electrical storage device in response to detecting a pulse of the radio frequency signal. When the discriminator detects the electrical signal, a trigger signal is produced, which causes the pulse circuit to apply the stimulation voltage pulse across the pair of electrodes.

### BRIEF DESCRIPTION OF THE DRAWINGS

FIG. 1 is a representation of a cardiac pacing apparatus attached to a medical patient;

FIG. 2 is a circuit diagram of a power transmitter for the cardiac pacing apparatus;

FIG. 3 is an isometric cut-away view of cardiac blood vessels in which a intravascular stimulator and a second electrode have been implanted;

FIG. 4 is a block diagram of an electrical circuit on the intravascular stimulator shown in FIG. 2; and

FIGS. 5 A, B, and C are waveform diagrams of three electrical signals in the cardiac pacing apparatus.

### DETAILED DESCRIPTION OF THE INVENTION

Although the present invention is being described in the context of cardiac pacing and of implanting a stimulator in a vein or artery of the heart, the present apparatus can be employed to stimulate of the areas of the human body. In addition to cardiac applications, the stimulation apparatus can provide brain stimulation, for treatment of Parkinson's disease or obsessive/compulsive disorder for example. The transvascular electrical stimulation also may be applied to muscles, the spine, the gastro/intestinal tract, the pancreas, and the sacral nerve. The apparatus may also be used for GERD treatment, endotracheal stimulation, pelvic floor stimulation, treatment of obstructive airway disorder and apnea, molecular therapy delivery stimulation, chronic constipation treatment, and electrical stimulation for bone healing.

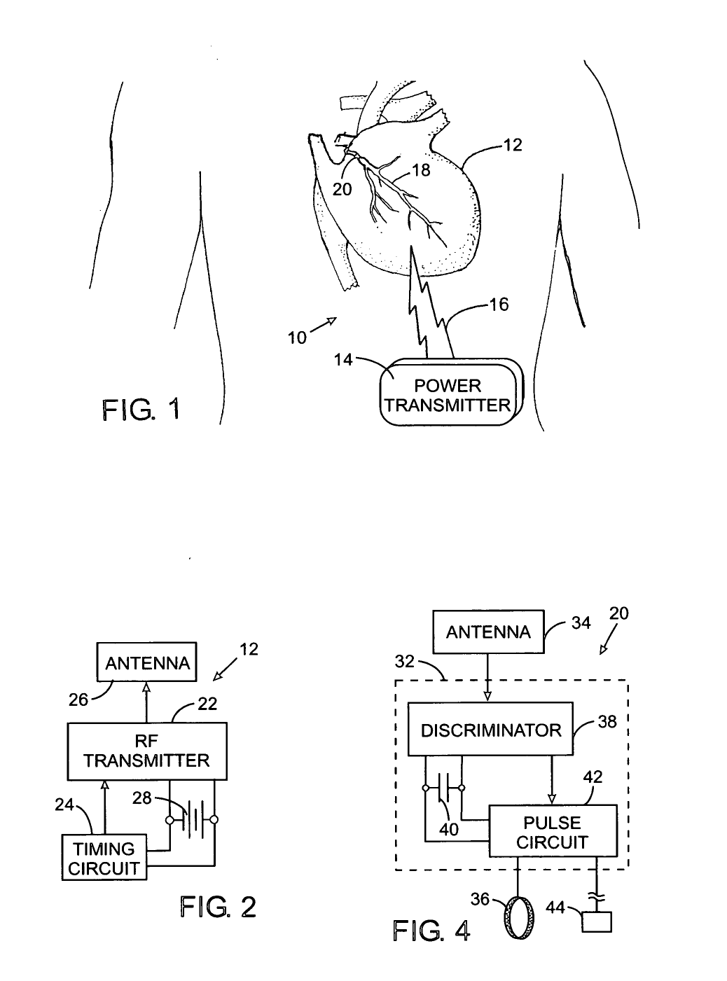

With initial reference to FIG. 1, a pacing apparatus 10 for electrically stimulating a heart 12 to contract comprises a power transmitter 14 and a intravascular stimulator 20. The power transmitter 14 preferably is worn outside the patient's body adjacent the chest and emits a radio frequency signal 16 which is received by the intravascular stimulator 20. Alternatively, the power transmitter 14 may be implanted in the patient. As will be described in greater detail, receipt of radio frequency signal 16 provides electrical power for circuitry on the stimulator. The intravascular stimulator 20 is placed in an artery or vein 18 in close proximity to the atria or ventricles. For example the intravascular stimulator 20 may be positioned in the coronary sinus vein.

Referring to FIG. 2, the power transmitter 14 comprises a radio frequency (RF) transmitter 22 connected to a timing circuit 24 and to an antenna 26. Both the RF transmitter 22 and the timing circuit 24 are powered by a battery 28. The timing circuit 24 controls the RF transmitter 22 to emit periodic pulses of the radio frequency signal 16. For example, the pulses have relatively slow rising and falling edges, as shown in FIG. 5A, so that the signal level gradually increases and decreases.

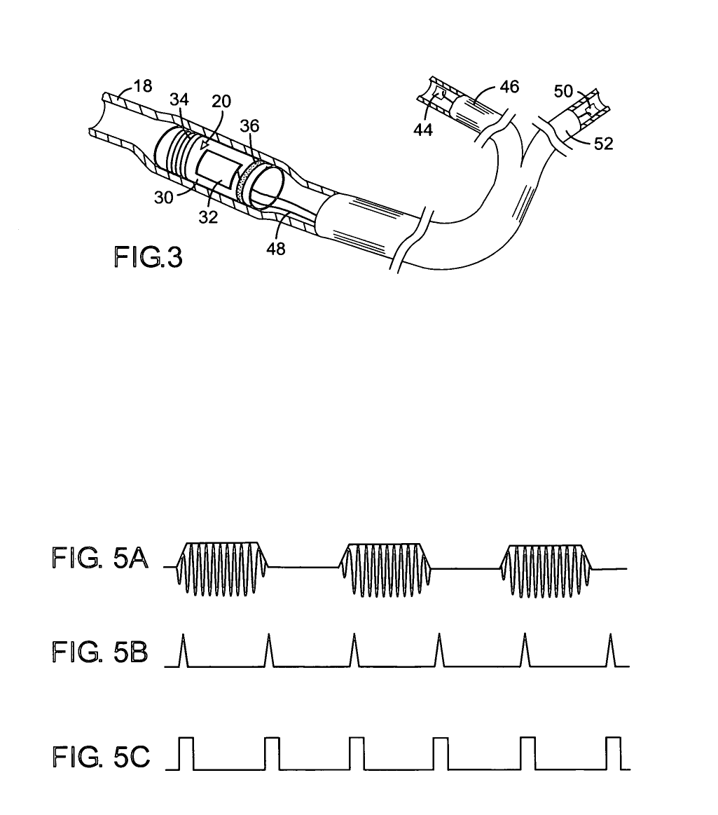

As illustrated in FIG. 3, the intravascular stimulator 20 includes a body 30 similar to well-known expandable vascular stents that are employed to enlarge a restricted vein or artery. However the stimulator body 30 merely has to engage the wall of the vein or artery to hold the stimulator in place and doe not have to enlarge the blood vessel. Such vascular stents have a generally tubular shape that initially is collapsed to a relatively small diameter enabling them to pass freely through blood vessels of a patient. The procedure for implanting the intravascular stimulator 20 is similar to that used for conventional vascular stents. For example, a balloon at the end of a standard catheter is inserted into the intravascular stimulator 20 in a collapsed configuration. That assembly is inserted through an incision in a vein or artery near the skin of a patient and passed through the vascular system to the appropriate location proximate to the atria or ventricles of the heart 12. The balloon of the catheter then is inflated to expand the intravascular stimulator 20, thereby slightly enlarging the blood vessel 18 which embeds the stimulator body 30 in the wall of the vein or artery. The balloon is deflated, the catheter is removed from the patient, and the incision is closed. Alternatively, a self-expanding stimulator body may be utilized. The tubular design of the body 30 allows blood to flow relatively unimpeded through the intravascular stimulator 20.

With reference to FIGS. 3 and 4, the intravascular stimulator 20 has a stimulation signal circuit 32 and a pickup device 34 in the form of a wire coil wound circumferentially around the body 30. A first electrode 36 in the form of a ring encircles the body. The stimulation signal circuit 32 includes a pulse discriminator 38 connected to the pickup device 34. As will be described, the pulse discriminator 38 distinguishes between electrical pulses induced in the pickup device 34 by electrical activity in the heart 12 and by the RF signal 16 from the power transmitter 14. That distinguishing is based on the shape of the respective signal waveform and the pulses of those waveforms as illustrated in FIG. 5A for the RF signal 16 and in FIG. 5B for the cardiac signal produced by activity of muscle fibers of the atria or ventricles. The cardiac signal that is detected may also originate in the sinus node of the heart 12. The RF signal has relatively long duration pulses with gradually rising and falling edges. In contrast, the electrical pulses of the cardiac signal are very short duration and rise and fall quickly. The pulse discriminator 38 also is able to detect when both types of pulses coincide in time.

Whenever an RF signal pulse is detected, the pulse discriminator 38 uses the energy of that signal to charge a storage capacitor 40 which supplies electrical power to the circuitry on the intravascular stimulator 20. Other types of electrical storage devices may be employed. The radio frequency signal supplies power to the intravascular stimulator, and unlike prior wireless pacemakers does not trigger cardiac stimulation.

The sinus node of the heart 12 emits an electrical cardiac signal which causes contraction of the heart chambers. The cardiac signal travels from cell to cell in paths through the heart to muscles which contract the atria. This signal also propagates along another path until reaching the atrioventricular (AV) node, which is a cluster of cells situated in the center of the heart between the atria and ventricles. The atrioventricular node serves as a gate that slows the electrical current before the cardiac signal is permitted to pass to the ventricles. This delay ensures that the atria have a chance to fully contract before the ventricles are stimulated. the resultant contraction of the cardiac muscle fibers also produces a cardiac signal.

Due to the placement of the intravascular stimulator 20 in proximity to the atrium or ventricle muscles, emission of the cardiac signal from that muscle fiber also induces an electric current pulse in the pickup device, or coil, 34 of the intravascular stimulator 20, as depicted in FIG. 5B. The pulse discriminator 38 recognizes the rapid rise time of this pulse as being produced by the cardiac signal, as compared to a RF signal pulse shown in FIG. 5A. When a cardiac signal pulse is detected, the pulse discriminator 38 issues a trigger signal to a pulse circuit 42. The pulse circuit 42 is similar to circuits used in previous cardiac pacing devices which generate voltage pulses for stimulating a contraction of the heart, as shown in FIG. 5C. Specifically, upon being triggered the pulse circuit 42 uses the charge on the capacitor 40 to produce a voltage pulse that is applied between the first electrode 36, that extends around the stimulator body 30, and a second electrode 44, which is remote from the intravascular stimulator 20.

As shown in FIG. 3, the second electrode 44 is adjacent to the wall of a blood vessel 46 in another section of the heart and is connected to the pulse circuit 42 by a thin insulated wire 48 extending through the blood vessels. The relatively small size of the second electrode 44 allows it to be placed into a significantly smaller blood vessel 46 than the intravascular stimulator 20. As a result, the second electrode 44 can be placed is a greater variety of locations in the cardiac vascular system and in close proximity to the muscles that contract the desired portion of the heart 12.

Depending upon whether the second electrode 44 is placed to stimulate contraction of an atrium or a ventricle, the pulse circuit 42 delays a predefined amount of time after receiving the trigger signal from the pulse discriminator 38 before applying the voltage pulse to the first and second electrodes. Therefore, timing of muscle stimulation corresponds to that which occurs with respect to naturally induced contraction of the atrium or ventricle. The duration of that delay is programmed into the pulse circuit 42.

In another version of the intravascular stimulator 20, one or more additional electrodes, such as a third electrode 50, can be implanted in other cardiac blood vessels 52 to stimulate further sections of the heart. In this case, individual voltage pulses can be applied between the first electrode 36 and each of the additional electrodes 44 and 50 to separately stimulate contraction of those other sections of the heart. A stimulation pulse also may be applied between the second and third electrodes 44 and 50, without using the first electrode 36.

The foregoing description was primarily directed to preferred embodiments of the invention. Even though some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

We claim:

1. An apparatus for artificially stimulating internal tissue of an animal, said apparatus comprising:
a power transmitter which periodically transmits a pulse of a radio frequency signal;
a first electrode and a second electrode for implantation into the animal; and
an intravascular stimulator for implantation in a blood vessel of the animal and comprising a body, a pickup device on the body for receiving the radio frequency signal, and a stimulation signal circuit on the body and connected to the pickup device, the stimulation signal circuit having an electrical storage device, wherein the stimulation signal circuit charges the electrical storage device with electrical energy from the radio frequency signal applies a stimulation voltage pulse across the first electrode and the second electrode to stimulate the internal tissue adjacent the blood vessel.

2. The apparatus as recited in claim 1 wherein the first electrode is mounted on the body of the intravascular stimulator.

3. The apparatus as recited in claim 2 wherein the second electrode is mounted on the body of the intravascular stimulator.

4. The apparatus as recited in claim 2 wherein the second electrode is remote from the body of the intravascular stimulator.

5. The apparatus as recited in claim 1 wherein the electrical storage device is a capacitor.

6. The apparatus as recited in claim 1 wherein the pickup device comprises a coil.

7. The apparatus as recited in claim 1 wherein the stimulation signal circuit comprises:
a discriminator connected to the pickup device, and charging the electrical storage device in response to detecting a pulse of the radio frequency signal, and producing a trigger signal; and
a pulse circuit connected to the discriminator and the electrical storage device, and applying the stimulation voltage pulse across the first electrode and the second electrode in response to the trigger signal.

8. The apparatus as recited in claim 8 wherein the pickup device also receives an electrical signal produced within the animal, and the discriminator distinguishes between the radio frequency signal from the power transmitter and electrical signal based on differences in their signal waveforms.

9. The apparatus as recited in claim 7 wherein each pulse of the radio frequency signal from the power transmitter has a leading edge which is longer in duration than a leading edge of the electrical signal produced within the animal.

10. The apparatus as recited in claim 1 wherein the pulses of the radio frequency signal from the power transmitter and pulses of the electrical signal produced within the animal are asynchronous.

11. The apparatus as recited in claim 1 further comprising a third electrode for implantation in the animal and connected to the intravascular stimulator, wherein the stimulation signal circuit applies a voltage pulse to the third electrode.
